(19) United States
(12) Patent Application Publication
(10) Pub. No.: US 2005/0214384 A1
(43) Pub. Date: Sep. 29, 2005

(54) Chromium compositions and methods for using the same for inhibiting drug-induced insulin resistance

(76) Inventors: Vijaya Juturu, Dobbs Ferry, NY (US); James R. Komorowski, Trumbell, CT (US); Gail Montgomery, Bedford, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/509,487

(22) PCT Filed: Apr. 3, 2003
(86) PCT No.: PCT/US03/10717

§ 371 Date: Sep. 27, 2004

Related U.S. Application Data
(60) Provisional application No. 60/375,848, filed on Apr. 23, 2002.

(51) Int. Cl. A61K 31/555; A61K 35/72; A61K 33/24

(52) U.S. Cl. 424/655; 514/184; 424/195.16

(57) ABSTRACT

A method for inhibiting drug-induced insulin resistance is provided which includes administering a dietary chromium complex to an individual receiving a contemporaneous dose of a drug that induces insulin resistance, wherein the amount of chromium complex administered is an amount effective to inhibit the development of insulin resistance. Advantageously, the amount of chromium complex administered per day is between about 300 and 1,000 micrograms per day. Compositions including a drug which induces insulin resistance in combination with a chromium complex are similarly described.

### BACKGROUND OF THE INVENTION

1. Field of the Invention

The present invention relates to the inhibition of drug-induced insulin resistance in an individual. More specifically, the invention relates to methods and compositions for reducing the incidence of drug-induced insulin resistance through chromium supplementation.

2. Description of the Related Art

Insulin Resistance Resulting from Certain Drug Therapies

Insulin resistance is a condition that is characterized by decreased insulin function and hyperinsulinemia. Individuals who have insulin resistance also have an increased risk of developing diabetes mellitus, dyslipidemia, hypertension, atherosclerosis, endothelial dysfunction, microalbuminuria, obesity, depression, Syndrome X, and polycystic ovary syndrome, among other conditions. In addition, all of the aforementioned conditions carry the risk of developing associated diseases. For example, diabetes increases the risk of developing associated diseases such as diabetic nephropathy, neuropathy, and retinopathy.

Insulin resistance may result from taking certain drug therapies such as statins, non-steroidal anti-inflammatory drugs (NSAIDS), steroids, oral contraceptives, hormone replacement therapy (HRT), beta blockers, potassium channel openers, diuretics, immunosuppressive drugs, etc. For example, A. Jula et al. report that fasting serum insulin levels increased 13%. and insulin resistance increased by 14% in 120 nondiabetic hypercholesterolemic male patients taking statin drugs to reduce their cholesterol levels (A. Jula et al., Effects of Diet and Simvastatin on Serum Lipids, Insulin, and Antioxidants in Hypercholesterolemic Men, 287 JAMA 598-605, 604 (2002)). Furthermore, it has also been reported that beta blockers and diuretics worsen insulin resistance and that patients taking beta blockers had a 28% higher incidence of diabetes than untreated patients with hypertension (S. Julius et al., Antihypertensive Treatment of Patients With Diabetes and Hypertension, 14 Am. J. Hypertens. 310S-316S, 313S (2001)).

Insulin resistance has also been described as a side effect of a variety of oral contraceptives. In a study of the metabolic effects of implantable steroid contraceptives, altered glucose tolerance characterized by decreased insulin sensitivity following glucose administration with implantable contraceptive brands such as Norplant®, Jadelle®, and Implanon® has been reported. (Dorfgliner, L. J., Metabolic Effects of Implantable Steroid Contraceptives for Women, 65 Contraception 47-62 (2002). See also, Peterson, K. R., Pharmacodynamic Effects of Oral Contraceptive Steroids on Biochemical Markers for Arterial Thrombosis, 49 Danish Medical Bulletin 43-60 (2002)). Similarly, oral contraceptives and hormone replacement therapy (“HRT”) have been linked to the onset of microalbuminuria. (Monster, T. B. M et al., Oral Contraceptive Use and Hormone Replacement Therapy Are Associated With Microalbuminuria, 161 Arch Intern Med. 2000-2005 (2001)).

When a patient develops insulin resistance, the physician will normally prescribe a hypoglycemic drug such as metformin, which the patient must continue to take for the rest of the patient's life.

The Role of Chromium

Dietary supplementation of chromium to normal individuals has been reported to lead to improvements in glucose tolerance, serum lipid concentrations, including high-density lipoprotein cholesterol, insulin and insulin binding (Anderson, 4 Clin. Psychol. Biochem. 31-41 1986). Supplemental chromium in the trivalent form, e.g. chromic chloride, is associated with improvements of risk factors associated with adult-onset (Type 2) diabetes and cardiovascular disease.

Chromium is a nutritionally essential trace element. The essentiality of chromium in the diet was established in 1959 by Schwartz, as cited in Present Knowledge in Nutrition, page 571, fifth edition (1984, the Nutrition Foundation, Washington, D.C.). Chromium depletion is characterized by the disturbance of glucose, lipid and protein metabolism and by a shortened lifespan. Chromium is essential for optimal insulin activity in all known insulin-dependent systems (Boyle et al., 70 Southern Med. J. 1449-1453 (1977)). Insufficient dietary chromium has been linked to both maturity-onset diabetes and to cardiovascular disease.

The principal energy sources for the body are glucose and fatty acids. Chromium depletion results in biologically ineffective insulin and compromised glucose metabolism. Under these conditions, the body must rely primarily on lipid metabolism to meet its energy requirements, resulting in the production of excessive amounts of acetyl-CoA and ketone bodies. Some of the documented acetyl-CoA is diverted to increased cholesterol biosynthesis, resulting in hypercholesterolemia. Diabetes mellitus is characterized in large part by glycosuria, hypercholesterolemia, and often ketoacidosis. The accelerated atherosclerotic process seen in diabetics is associated with hypercholesterolemia (Boyle et al., supra.).

Chromium functions as a cofactor for insulin. It binds to the insulin receptor and potentiates many, and perhaps all, of its functions (Boyle et al., supra.). These functions include, but are not limited to, the regulation of carbohydrate and lipid metabolism. (Present Knowledge in Nutrition, supra, at p. 573-577). The introduction of inorganic chromium compounds per se into individuals is not particularly beneficial. Chromium must be converted endogenously into an organic complex or must be consumed as a biologically active molecule. Only about 0.5% of ingested inorganic chromium is assimilated into the body (Recommended Daily Allowances, Ninth Revised Edition, The National Academy of Sciences, page 160, 1980). Only 1-2% of most organic chromium compounds are assimilated into the body.

U.S. Pat. No. Re. 33,988 discloses that when selected essential metals, including chromium, are administered to mammals as exogenously synthesized coordination complexes of picolinic acid, they are directly available for absorption without competition from other metals. This patent describes a composition and method for selectively supplementing the essential metals in the human diet and for facilitating absorption of these metals by intestinal cells. These complexes are safe, inexpensive, biocompatible, and easy to produce. These exogenously synthesized essential metal coordination complexes of picolinic acid (pyridine-2-carboxylic acid) have the following structural formula: 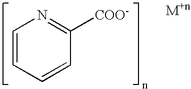
wherein M represents the metallic cation and n is equal to the cation's valence. For example, when M is Cr and n=3, then the compound is chromic tripicolinate. Other chromium picolinates disclosed include chromic monopicolinate and chromic dipicolinate.

The U.S. Recommended Daily Intake (RDI) of chromium is 120 μg. U.S. Pat. No. 5,087,623, describes the administration of chromic tripicolinate for the treatment of adult-onset diabetes in doses ranging from 50 to 500 μg. U.S. Pat. No. 6,329,361, discloses the use of high doses of chromic tripicolinate (providing 1,000-10,000 μg chromium/day) for reducing hyperglycemia and stabilizing the level of serum glucose in humans with Type 2 diabetes. U.S. Pat. Nos. 5,789,401 and 5,929,066, disclose a chromic tripicolinate-biotin composition and its use in lowering blood glucose levels in humans with Type 2 diabetes.

U.S. Pat. Nos. 5,087,623; 5,087,624; and 5,175,156, disclose the use of chromium tripicolinate for supplementing dietary chromium, reducing hyperglycemia and stabilizing serum glucose, increasing lean body mass and reducing body fat, and controlling blood serum lipid levels, including the lowering of undesirably high blood serum LDL-cholesterol levels and the raising of blood serum High Density Lipid (HDL)-cholesterol levels, the so-called “good” cholesterol. U.S. Pat. Nos. 4,954,492 and 5,194,615, describe a related complex, chromic nicotinate, which is also used for supplementing dietary chromium and lowering serum lipid levels. Picolinic acid and nicotinic acid are position isomers having the following structures:

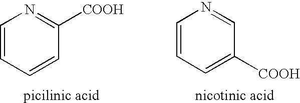

Nicotinic acid and picolinic acid form coordination complexes with monovalent, divalent and trivalent metal ions and facilitate the absorption of these metals by transporting them across intestinal cells and into the bloodstream. Chromium absorption in rats following oral administration of CrCl₃ was facilitated by the non-steroidal anti-inflammatory drugs (NSAIDs) aspirin and indomethacin (Davis et al., 15 J. Nutrition Res. 202-210 (1995); Kamath et al., 127 J. Nutrition 478-482 (1997)). These drugs inhibit the enzyme cyclooxygenase which converts arachidonic acid to various prostaglandins, resulting in inhibition of intestinal mucus formation and lowering of intestinal pH which facilitates chromium absorption.

U.S. Pat. No. 4,315,927 discloses that when selected essential metals are administered to mammals as exogenously synthesized coordination complexes of picolinic acid, they are directly available for absorption without competition from other metals. These complexes are safe, inexpensive, biocompatible and easy to produce.

It would be desirable for patients not to develop insulin resistance as a side effect of taking drugs to treat other medical conditions such as dyslipidemia, hypertension, etc. There is a constant need for effective methods of inhibiting the onset of drug-induced insulin resistance. The present invention addresses this need by providing a safe, inexpensive, drug-free therapeutic agent.

### SUMMARY OF THE INVENTION

The present invention is directed to inhibiting the onset of drug-induced insulin resistance in an individual. Accordingly, in one aspect of the invention, a method for inhibiting the development of drug-induced insulin resistance including administering a dietary chromium complex to an individual receiving a contemporaneous dose of a drug that induces insulin resistance is provided. Advantageously, the amount of chromium complex administered is an amount effective to inhibit the development of insulin resistance.

In one aspect of the invention, the drug that induces insulin resistance may be a statin drug, non-steroidal anti-inflammatory drug, steroid, oral contraceptive, hormone replacement therapy drug, beta blocker, potassium channel opener, or diuretic.

Generally, the effective dose of chromium provided by the chromium complex is at least 50 μg per day. The chromium complex may be a trivalent chromium complex such as chromium picolinate, chromic tripicolinate, chromium nicotinate, chromic polynicotinate, chromium chloride, chromium histidinate, chromium yeast, or any other chromium complex, whether now known or to be developed in the future

Preferably, the chromium complex is in a pharmaceutically acceptable carrier.

Optionally, the chromium complex is orally administered. However, in some aspects of the invention, the chromium complex is parenterally administered.

In yet another aspect of the invention, certain chelating agents may be added to facilitate absorption of the chromium complex. Optionally, the ratio of the chromium complex to the chelating agent is between about 10:1 to about 1:10 (w/w). In one aspect of the invention, picolinic acid is administered to an individual. In another aspect, nicotinic acid is administered to an individual. In still another aspect, both picolinic and nicotinic acid are administered to an individual in order to inhibit the onset of drug-induced insulin resistance.

In still another aspect of the invention, the chromium complex and the drug that induces insulin resistance are administered simultaneously. In another aspect, the chromium complex is administered within 24 hours of the drug that induces insulin resistance.

In yet another aspect of the invention, the method of inhibiting drug-induced insulin resistance includes administering an effective dose of a hypoglycemic drug such as metformin, sulfonylureas, and glitazones.

In another aspect of the invention, compositions comprising an effective pharmacological amount of a drug which induces insulin resistance in combination with a sufficient amount of a chromium complex to inhibit the onset of insulin resistance are provided. The chromium complex may include chromium picolinate, chromic tripicolinate, chromium nicotinate, chromic polynicotinate, chromium chloride, chromium histidinate, chromium yeast, or other chromium complex, whether now known or to be developed in the future. Preferably, the sufficient amount of chromium provided by the chromium complex and contained in the composition is between about 50 μg and 2000 μg.

### DETAILED DESCRIPTION OF THE PREFERRED EMBODIMENT

Numerous drug therapies have been implicated in causing drug-induced insulin resistance. For example, the use of statins, non-steroidal anti-inflammatory drugs (NSAIDS), steroids, oral contraceptives, hormone replacement therapy (HRT), beta blockers, potassium channel openers, and diuretics have been linked to an increased incidence of insulin resistance. At the present time, no drug or treatment exists or has been suggested to inhibit the onset of drug-induced insulin resistance. Instead, drugs have been formulated to treat drug-induced resistance once it has occurred.

The present invention is based, in part, on the novel and unexpected discovery that when an individual is administered a chromium complex concomitantly with certain drugs which cause drug-induced insulin resistance, the symptoms and incidence of insulin stance is lowered. Accordingly, in one embodiment, a method for the inhibition of drug-induced insulin resistance including chromium supplementation is provided. Compositions for the inhibition of drug-induced insulin resistance in an individual are similarly provided.

The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the invention herein described. As used herein, the term “chromium complexes” or “chromium complex” includes, without limitation, all trivalent chromium complexes, such as chromium picolinate, chromic tripicolinate, chromium nicotinate, chromic polynicotinate, chromium chloride, chromium histidinate, chromium yeast, and other chromium complexes, whether now known or developed in the future.

As used herein, the phrase “drug which induces insulin resistance” means any substance which may induce insulin resistance when administered to a human or other animal. Examples of drugs which induce insulin resistance include, without limitation, statin drugs such as simvastatin, cerivastatin, pravastatin, atorvastatin, fluvastatin, and lovastatin; non-steroidal anti-inflammatory drugs such as cimicifuga, choline salicylate-magnesium salicylate, diclofenac sodium, diclofenac potassium, diflunisal, etodolac, fenoprofen calcium, floctafenine, flurbiprofen, ibuprofen, indomethacin, ketoprofen, ketorolac tromethamine, magnesium salicylate, mefenamic acid, nabumetone, naproxen, naproxen sodium, oxyphenbutazone, phenylbutazone, piroxicam, salsalate, sodium salicylate, sulindac, tenoxicam, taiprofenic acid, and tolmetin sodium; steroids such as hydrocortisone, dexamethasone, and methylprednisolone; contraceptives including oral contraceptives such as estrogen, progesterone and progestin as well as implantable contraceptives such as levonorgestrel, etonogestrel, nomegestrol acetate, and nestorone; hormone replacement therapy (HRT) drugs including conjugated equine estrogens, esterified estrogens, estradiol, estrone, synthetic conjugated estrogens, estropipate, estropipate, ethinyl estradiol, norethindrone, medroxyprogesterone acetate, progestin, natural progesterone, tamoxifen, testosterone, and raloxifene; beta blocker drugs including acebutolol, atenolol, betaxolol, bucinodol, carteolol, labetalol, metoprolol, nadolol, penbutolol, pindolol, propanolol, and timolol; and diuretics. Three primary types of diuretics exist which include thiazides, loop diuretics, and potassium sparing agents. As used herein, the term “diuretic” or “diuretics” includes, without limitation, hydrochlorothiazide, chlorthalidone, chlorothiazide, indapamide, metolazone, amiloride, spironolactone, triamterene, furosemide, bumetanide, ethacrynic acid, and torsemide. Certain immunosuppressive drugs such as prednisolone, cyclosporin A, and tacromlimus and potassium channel modulators such as nicorandil are also included in the definition of drugs which induce insulin resistance. The above list is provided for example purposes only and it is understood that the definition of “drug which induces insulin resistance” includes those drugs which induce insulin resistance that are not specifically listed above, as well as those drugs which are found to induce insulin resistance, whether in existence today or developed in the future.

The administration of an effective dose of a chromium complex to subjects who are taking drugs which have been linked with the onset of insulin resistance actually inhibits or attenuates the onset of insulin resistance. The supplementation with a chromium complex to a subject taking a drug which induces insulin resistance results in a lowered incidence of drug-induced insulin resistance. By not developing insulin resistance in the first place, the patient is not exposed to the associated diseases and risks. The patient also does not need to take additional, and sometimes costly, medications to treat the insulin resistance and associated diseases.

Without being limited to a particular theory, we propose that chromium supplementation inhibits drug-induced insulin resistance from developing by reducing fasting insulin levels and lowering blood sugar. Accordingly, in one embodiment, a method of inhibiting drug-induced insulin resistance through chromium supplementation is provided.

Chromium supplementation includes the administration of any chromium complex or combination of chromium complexes to an individual who is concurrently being administered a drug which induces insulin resistance. Advantageously, the chromium complexes are synthetic. The synthesis and use of chromium picolinates, for example, is described in U.S. Pat. Nos. Re 33,988 and 5,087,623. Chromic tripicolinate is available from health food stores, drug stores and other commercial sources. The synthesis and use of chromic polynicotinate is described in U.S. Pat. No. 5,194,615.

The amount of chromium necessary to obtain the desired effect, i.e., to thwart the development of insulin resistance, will depend on the particular insulin-resistance-inducing-drug and dosage of such drug that the subject is required to take. In general, the level of chromium used for supplementation in order to inhibit the onset of drug-induced insulin resistance is at least about 50 μg/day. Note in particular that chromium picolinate and chromium chloride have been administered to rats at levels several thousand times the upper limit of the estimated safe and adequate daily dietary intake (ESADDI) for chromium for humans (based on body weight) without toxic effects. R. Anderson et al., Lack of Toxicity of Chromium Chloride and Picolinate, 16 J. Am. Coll. Nutr. 273-279 (1997). While the level of chromium used for supplementation may be within several thousand times the upper limit of the ESADDI, preferably, the amount of chromium is between about 50 and 2,000 μg/day. More preferably, the amount of chromium is between about 300 and 1,000 μg/day. Most preferably, the amount of chromium is between about 400 and 1,000 μg/day. In a particularly preferred embodiment, the amount of chromium is between about 600 and 1,000 μg/day. Note that these doses are based on a 70 kg adult human, and that the dose can be applied on a per-kilogram basis to humans or animals of different weights.

Inhibition of drug-induced insulin resistance is accomplished by administering a drug which induces insulin resistance and an effective dose of a chromium complex to an individual separately or as a single composition. A subject may begin chromium supplementation at the beginning of their treatment with insulin-resistance-inducing-drugs. Alternatively, the subject may begin supplementation with a chromium complex after the subject's treatment with insulin-resistance-inducing-drugs has begun, but before developing insulin resistance.

Advantageously, an individual is administered a pharmaceutically effective dose of a chromium complex such as chromium picolinate. In one embodiment, the drug which induces insulin resistance and chromium complex are administered substantially simultaneously. In an alternative embodiment, the chromium complex is administered first and then the drug which induces insulin resistance is added second. In yet another embodiment, the drug which induces insulin resistance is administered first. If administered separately, the chromium complex and drug which induces insulin resistance should be given in a temporally proximate manner, e.g. within a twenty-four hour period, such that the inhibition of drug-induced insulin resistance is enhanced. More particularly, the chromium complex and drug which induces insulin resistance may be given within one hour of each other. In one embodiment, the drug which induces insulin resistance is prepared as a single formulation to include both the active ingredient of the drug and an effective dose of a chromium complex. One of skill in the art will appreciate that other components may be added separately or incorporated into a single formulation to enhance the effects of chromium in inhibiting drug-induced insulin resistance. As will be described in greater detail below, uncomplexed chelating agents such as nicotinic acid, picolinic acid, or both nicotinic and picolinic acids can be included in the formulation or added separately to enhance the absorption of the chromium complex.

While the chromium complexes aid in the absorption of chromium by intestinal cells, in some embodiments, uncomplexed chelating agents are advantageously included in the compositions to facilitate absorption of other ingested chromium as well as other metals including, but not limited to, copper, iron, magnesium, manganese, and zinc. Suitable chelating agents include picolinic acid, nicotinic acid, or both picolinic acid and nicotinic acid. Thus, the compositions of the disclosed invention are readily absorbable forms of chromium which also facilitate absorption of other essential metals in the human diet.

The chelating agents such as picolinic acid and nicotinic acid are available from many commercial sources, including Sigma-Aldrich (St. Louis, Mo.) (picolinic acid; catalog No. P5503; nicotinic acid; catalog No. PN4126). Preferably, the ratio of the chromium complex to the chelating agent from about 10:1 to about 1:10 (w/w), more preferably from about 5:1 to about 1:5 (w/w). Alternatively, the molar ratio of chromium complex to the uncomplexed chelating agent is preferably 1:1, and may be from about 5:1 to about 1:10.

The administration of chromium can be by any of the methods of administration described below or by drug delivery methods known by one of skill in the art. The compositions may be administered orally, through parenteral nutrition, e.g., feeding tube or intravenously, and through other known means. Chromium picolinate is particularly preferred as the source of chromium supplementation due to its high level of bioavailability, but any form of dietary chromium may be used.

For oral administration, the chromium complex may be provided as a tablet, aqueous or oil suspension, dispersible powder or granule, emulsion, hard or soft capsule, syrup, elixir, or beverage. Compositions intended for oral use may be prepared according to any method known in the art for the manufacture of pharmaceutically acceptable compositions and such compositions may contain one or more of the following agents: sweeteners, flavoring agents, coloring agents and preservatives. The sweetening and flavoring agents will increase the palatability of the preparation. Tablets containing chromium complex in admixture with non-toxic pharmaceutically acceptable excipients suitable for tablet manufacture are acceptable. Pharmaceutically acceptable means that the agent should be acceptable in the sense of being compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets may be uncoated or may be coated by known techniques to delay disintegration and absorption in the gastrointestinal tract and thereby provide a sustained action over a longer period of time. For example, a time delay material such as glyceryl monostearate or glyceryl distearate alone or with a wax may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. Aqueous suspensions may contain the chromium complex of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

The chromium complex preparations for parenteral administration may be in the form of a sterile injectable preparation, such as a sterile injectable aqueous or oleaginous suspension. This suspension may be formulated according to methods well known in the art using suitable dispersing or wetting agents and suspending agents. The sterile injectable preparation may also be a sterile injectable solution or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils may be employed conventionally as a solvent or suspending medium. For this purpose, any bland fixed oil may be employed including synthetic mono or diglycerides. In addition, fatty acids such as oleic acid may likewise be used in the preparation of injectable preparations.

The pharmaceutical compositions may also be in the form of oil-in-water emulsions. The oily phase may be a vegetable oil, such as olive oil or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

It will be appreciated by the skilled artisan that the amount of chromium complex that may be combined with the carrier material to produce a single dosage form will vary depending upon the host treated and the particular mode of administration.

Insulin resistance is a key pathogenic parameter of Type 2 diabetes, and clinical interventions that improve insulin sensitivity are considered cornerstones in the management of the disease. In addition, the relationship of insulin resistance to cardiovascular disease and its associated risk factors has been well established over the past few years. Therefore, in a preferred embodiment, methods and compositions for thwarting the development of insulin resistance are provided comprising the administration of a chromium complex and a hypoglycemic drug such as metformin inhibit insulin resistance from developing. Combinations of pharmacologic agents (such as sulfonylureas/metformin, sulfonylureas/glitazones, and metformin/glitazones) are highly effective pharmacologic interventions that appear to lower both glucose and insulin levels. Further, there is evidence that triple drug therapy (e.g. sulfonylureas/metformin/glitazones) can lower clinical glycemia in addition to lowering insulin levels. Hence, in some embodiments, compositions comprising a chromium complex with metformin, sulfonylureas, and glitazones or combinations thereof are administered to a subject taking drugs which are induce insulin resistance to inhibit the onset of such insulin resistance.

The instant disclosure differs from the present technology in that the patient has a lesser chance of developing drug-induced insulin resistance. By not developing insulin resistance in the first place, the patient is not exposed to the associated diseases and risks. The patient also does not need to take additional, and sometimes costly, medications to treat the insulin resistance and associated diseases.

### EXAMPLES

The following examples teach the methods and compositions disclosed herein for inhibiting drug-induced insulin resistance through the administration of at least one chromium complex. These examples are illustrative only and are not intended to limit the scope of the invention disclosed herein. The treatment method described below can be optimized using empirical techniques well known to those of ordinary skill in the art. Moreover, artisans of skill would be able to use the teachings described in the following examples to practice the full scope of the invention disclosed herein.

### Example 1

### Study of the Effects of Chromium Picolinate on Inhibiting Statin-Induced Insulin Resistance

The effect of a chromium complex in inhibiting drug-induced insulin resistance is evaluated. Specifically, the effects of chromium picolinate on insulin sensitivity and vascular reactivity in subjects taking statin drugs are evaluated.

A clinical trial is initiated which includes approximately 80 male and female subjects between the ages of 35 and 65. The subject population is characterized as individuals suffering from moderate hypercholesterolemia and moderate high blood pressure. All subjects have been diagnosed with hypercholesterolemia. LDLC based on 75^{th} percentile (>140-165 mg/dL) and HDL-C: <30 mg/dL. Subjects who are insulin resistant and/or who possess triglyceride profiles of >400 mg/dL and/or blood glucose levels of >140 mg/dl are excluded from participation in the study.

All subjects are taking standard statin drugs for approximately six (6) months prior to the start of the investigation. None of the subjects included in the study use other cholesterol lowering drugs or other drugs such as beta-blockers, thiazide, diuretics, steroids, oral contraceptives, chromium or niacin supplements, or any investigational drugs.

None of the subjects included in the study have medical or surgical conditions such as diabetes, hypertension, subacute bacterial endocarditis (SB), hyperthyroid disease, renal failure, liver disease, diabetes mellitus, other metabolic disorders, known familial lipid disorders, alcohol or drug abuse, bleeding disorders, pregnancy, lactation, or any other medical condition which may interfere with the interpretation of the results from the study.

Study visits are scheduled at the start of the study to record baseline information on the subjects and every two weeks thereafter. The following physiological conditions are measured prior to administration of the supplement as a baseline and at regular intervals during the course of the study: Glycated hemoglobin, fasting insulin, fasting plasma glucose levels, total cholesterol, triglycerides, LDL, HDL, urinalysis (routine), CBC, and serum chemistry, as well as blood pressure and body weight. In addition, insulin sensitivity is measured according to the euglycemic-hyperinsulinemic glucose clamp technique specified by R. A. DeFronzo et al. Glucose Clamp Technique: A Method for Quantifying Insulin Secretion and Resistance, 237 Am. J. Physiol. E214-E223 (1979).

The subjects are divided into a two-way, randomized, double-blind, placebo-controlled, parallel group study. The two groups are chromium picolinate alone and placebo. Subjects are administered either chromium picolinate (400 μg chromium), or a placebo comprising calcium phosphate orally in the form of a capsule. The subjects take one capsule a day with a meal and do not know the contents of each capsule. Subjects are asked not to alter their dietary or exercise habits during the study. The duration of the study is approximately six months.

After the study is concluded, the data are analyzed and reveal that subjects who are administered chromium are observed to have a lower incidence of drug-induced insulin resistance than the subjects who are administered a statin drug without chromium supplementation. An inhibition of the onset of drug-induced insulin resistance is observed.

### Example 2

### Drug Formulation Including a Chromium Complex to Inhibit the Onset of Drug-Induced Insulin Resistance

Oral contraceptives have long been associated with glucose intolerance. Women who take oral contraceptives have an increased risk of developing drug-induced insulin resistance. Accordingly, it would be of great benefit to women's health to develop formulations of oral contraceptives with chromium complexes to thwart the development of drug-induced insulin resistance and the attendant diseases associated with insulin resistance such as arterial thrombosis, cardiovascular disease, diabetes, and hypercholesterolemia.

An effective pharmacological amount of an oral contraceptive is formulated in combination with chromium tripicolinate as a tablet. The tablet contains 75 μg of chromic tripicolinate. The oral contraceptive containing chromic tripicolinate has a lower incidence of causing drug-induced insulin resistance than those oral contraceptives which lack a chromium complex.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being redefined herein to be restricted to including any specific characteristics of the features or aspects of the invention with which that terminology is associated. The scope of the invention should therefore be construed in accordance with the appended claims and any equivalents thereof.

1. A method for inhibiting the development of drug-induced insulin resistance comprising:
administering a dietary chromium complex to an individual receiving a contemporaneous dose of a drug that induces insulin resistance, wherein the amount of chromium complex administered is an amount effective to inhibit the development of insulin resistance.

2. The method of claim 1, wherein said drug is selected from the group consisting of statins, non-steroidal anti-inflammatory drugs, steroids, oral contraceptives, hormone replacement therapy, beta blockers, potassium channel openers, immunosuppressants, and diuretics.

3. The method of claim 1, wherein the effective dose of chromium provided by said chromium complex is at least about 50 μg per day.

4. The method of claim 1, wherein said chromium complex is a trivalent chromium complex.

5. The method of claim 1, wherein said chromium complex is selected from the group consisting of chromium picolinate, chromic tripicolinate, chromium nicotinate, chromic polynicotinate, chromium chloride, chromium histidinate, and chromium yeasts.

6. The method of claim 1, wherein said chromium complex is in a pharmaceutically acceptable carrier.

7. The method of claim 1, wherein said chromium complex is orally administered.

8. The method of claim 1, wherein said chromium complex is parenterally administered.

9. The method of claim 1, further comprising administering to said individual a chelating agent.

10. The method of claim 9, wherein the ratio of the chromium complex to the chelating agent is between about 10:1 to about 1:10 (w/w).

11. The method of claim 9, wherein said chelating agent is picolinic acid, nicotinic acid, or a combination of both picolinic acid and nicotinic acid.

12. The method of claim 1, wherein said chromium complex and said drug that induces insulin resistance are administered simultaneously.

13. The method of claim 1, wherein said chromium complex is administered is administered within 24 hours of said drug that induces insulin resistance.

14. The method of claim 1, further comprising administering to said individual an effective dose of a hypoglycemic drug selected from the group consisting of metformin, sulfonylureas, and glitazones.

15. A composition comprising an effective pharmacological amount of a beta blocker drug in combination with a sufficient amount of a chromium complex to inhibit the onset of insulin resistance.

16. The composition of claim 15, wherein said beta blocker is selected from the group consisting of acebutolol, atenolol, betaxolol, bucinodol, carteolol, labetalol, metoprolol, nadolol, penbutolol, pindolol, propanolol, and timolol.

17. The composition of claim 15, wherein said chromium complex is selected from the group consisting of chromium picolinate, chromic tripicolinate, chromium nicotinate, chromic polynicotinate, chromium chloride, chromium histidinate, and chromium yeasts.

18. The composition of claim 15, wherein said sufficient amount of chromium provided by said chromium complex is at least about 50 μg.

19. A composition comprising an effective pharmacological amount of a contraceptive drug in combination with a sufficient amount of a chromium complex to inhibit the onset of insulin resistance.

20. The composition of claim 19, wherein said contraceptive drug is selected from the group consisting of estrogen, progesterone, progestin, levonorgestrel, etonogestrel, nomegestrol acetate, and nestorone.

21. The composition of claim 19, wherein said chromium complex is selected from the group consisting of chromium picolinate, chromic tripicolinate, chromium nicotinate, chromic polynicotinate, chromium chloride, chromium histidinate, and chromium yeasts.

22. The composition of claim 19, wherein said sufficient amount of chromium provided by said chromium complex is at least about 50 μg.

23. A position comprising an effective pharmacological amount of a statin drug in combination with a sufficient amount of a chromium complex to inhibit the onset of insulin resistance.

24. The composition of claim 23, wherein said statin drug is selected from the group consisting of simvastatin, cerivastatin, pravastatin, atorvastatin, fluvastatin, and lovastatin.

25. The composition of claim 23, wherein said chromium complex is selected from the group consisting of chromium picolinate, chromic tripicolinate, chromium nicotinate, chromic polynicotinate, chromium chloride, chromium histidinate, and chromium yeasts.

26. The composition of claim 23, wherein said sufficient amount of chromium provided by said chromium complex is at least about 50 μg.

27. A composition comprising an effective pharmacological amount of a non-steroidal anti-inflammatory drug in combination with a sufficient amount of a chromium complex to inhibit the onset of insulin resistance.

28. The composition of claim 27, wherein said non-steroid anti-inflammatory drug is selected from the group consisting of cimicifuga, choline, salicylate-magnesium salicylate, diclofenac sodium, diclofenac potassium, diflunisal, etodolac, fenoprofen calcium, floctafenine, flurbiprofen, ibuprofen, indomethacin, ketoprofen, ketorolac tromethamine, magnesium salicylate, mefenamic acid, nabumetone, naproxen, naproxen sodium, oxyphenbutazone, phenylbutazone, piroxicam, salsalate, sodium salicylate, sulindac, tenoxicam, taiprofenic acid, and tolmetin sodium.

29. The composition of claim 27, wherein said chromium complex is selected from the group consisting of chromium picolinate, chromic tripicolinate, chromium nicotinate, chromic polynicotinate, chromium chloride, chromium histidinate, and chromium yeasts.

30. The composition of claim 27, wherein said sufficient amount of chromium provided by said chromium complex is at least about 50 μg.

31. A composition comprising an effective pharmacological amount of a steroid drug in combination with a sufficient amount of a chromium complex to inhibit the onset of insulin resistance.

32. The composition of claim 31, wherein said steroid is selected from the group consisting of hydrocortisone, dexamethasone, and methylprednisolone.

33. The composition of claim 31, wherein said chromium complex is selected from the group consisting of chromium picolinate, chromic tripicolinate, chromium nicotinate, chromic polynicotinate, chromium chloride, chromium histidinate, and chromium yeasts.

34. The composition of claim 31, wherein said sufficient amount of chromium provided by said chromium complex is at least about 50 μg.

35. A composition comprising an effective pharmacological amount of a potassium channel opener in combination with a sufficient amount of a chromium complex to inhibit the onset of insulin resistance.

36. The composition of claim 35, wherein said potassium channel opener is nicorandil.

37. The composition of claim 35, wherein said chromium complex is selected from the group consisting of chromium picolinate, chromic tripicolinate, chromium nicotinate, chromic polynicotinate, chromium chloride, chromium histidinate, and chromium yeasts.

38. The composition of claim 35, wherein said sufficient amount of chromium provided by said chromium complex is at least about 50 μg.

39. A composition comprising an effective pharmacological amount of a diuretic in combination with a sufficient amount of a chromium complex to inhibit the onset of insulin resistance.

40. The composition of claim 39, wherein said diuretic is selected from the group consisting of hydrochlorothiazide, chlorthalidone, chlorothiazide, indapamide, metolazone, amiloride, spironolactone, triamterene, furosemide, bumetanide, ethacrynic acid, and torsemide.

41. The composition of claim 39, wherein said chromium complex is selected from the group consisting of chromium picolinate, chromic tripicolinate, chromium nicotinate, chromic polynicotinate, chromium chloride, chromium histidinate, and chromium yeasts.

42. The composition of claim 39, wherein said sufficient amount of chromium provided by said chromium complex is between about 50 μg.

43. A composition comprising an effective pharmacological amount of a hormone replacement therapy drug in combination with a sufficient amount of a chromium complex to inhibit the onset of insulin resistance.

44. The composition of claim 43, wherein said hormone replacement therapy drug is selected from the group consisting of conjugated equine estrogens, esterified estrogens, estradiol, estrone, synthetic conjugated estrogens, estropipate, estropipate, ethinyl estradiol, norethindrone, medroxyprogesterone acetate, progestin, natural progesterone, tamoxifen, testosterone, and raloxifene.

45. The composition of claim 43, wherein said chromium complex is selected from the group consisting of chromium picolinate, chromic tripicolinate, chromium nicotinate, chromic polynicotinate, chromium chloride, chromium histidinate, and chromium yeasts.

46. The composition of claim 43, wherein said sufficient amount of chromium provided by said chromium complex is between about 50 μg.

47. A method for inhibiting the development of a secondary disease resulting from insulin resistance that comprises:
administering a dietary chromium complex to an individual receiving a contemporaneous dose of a drug that induces insulin resistance, wherein the amount of chromium complex administered is an amount effective to inhibit the development of insulin resistance.

48. The method of claim 47, wherein the secondary disease is selected from the group consisting of atherosclerosis, hypertension, endothelial dysfunction, microalbuminuria, obesity, dyslipidemia, diabetes mellitus, depression, Syndrome X, polycystic ovary syndrome, diabetic nephropathy, diabetic neuropathy, and diabetic retinopathy.

49. The method of claim 48, wherein said chromium complex is selected from the group consisting of chromium picolinate, chromic tripicolinate, chromium nicotinate, chromic polynicotinate, chromium chloride, chromium histidinate, and chromium yeasts.

50. The composition of claim 49, wherein said sufficient amount of chromium provided by said chromium complex is between about 50 μg.
